(19) United States
(12) Patent Application Publication
(10) Pub. No.: US 2025/0058107 A1
(43) Pub. Date: Feb. 20, 2025

(54) INTEGRATION OF RELATED PROBABILITY SHELLS INTO DEEP BRAIN STIMULATION TARGETING

(71) Applicant: Boston Scientific Neuromodulation Corporation, Valencia, CA (US)

(72) Inventors: Richard Mustakos, Simi Valley, CA (US); Mahsa Malekmohammadi, Sherman Oaks, CA (US); Stephen Randolph Smith, III, North Hollywood, CA (US)

(73) Assignee: Boston Scientific Neuromodulation Corporation, Valencia, CA (US)

(21) Appl. No.: 18/804,678

(22) Filed: Aug. 14, 2024

Related U.S. Application Data
(60) Provisional application No. 63/533,038, filed on Aug. 16, 2023.

(51) Int. Cl. A61N 1/36; G16H 20/30; G16H 30/20

(52) U.S. Cl. CPC A61N 1/36031; G16H 20/30; G16H 30/20

(57) ABSTRACT

Methods and systems for analyzing and selecting therapy configurations for use in stimulating neural tissue. Probability shells relating to a likelihood that electrical stimulation issued to a given volume of neural tissue will cause a therapeutic outcome are integrated in a system for analyzing anatomical and other data, including lead position relative to neural anatomy. Metrics for analyzing therapy configurations can then be calculated, and the process of identifying likely beneficial therapy configurations is enhanced.

### CROSS REFERENCE TO RELATED APPLICATIONS

The present application claims the benefit of U.S. Provisional Patent Application No. 63/533,038, filed Aug. 16, 2023, titled INTEGRATION OF RELATED PROBABILITY SHELLS INTO DEEP BRAIN STIMULATION TARGETING, the disclosure of which is incorporated herein by reference.

### BACKGROUND

Deep brain stimulation (DBS) is a form of neuromodulation in which electrodes are implanted into the brain of a patient and used to deliver stimuli. Therapies directed to a variety of ailments, including Alzheimer's Disease, Parkinson's Disease, cognitive and/or memory decline, depression and other disorders have been proposed and/or implemented. Each patient has unique anatomy, and each treated ailment may call for different portions of the brain to receive therapy. As a result, accurate and precise targeting of therapy is desired. In many cases, the exact location of a targeted brain structure cannot be directly determined, but is inferred from combinations of imaging studies and population-based brain atlas data. A particular structure may be characterized as having a likelihood or probability of being located at a particular location. Likewise, a particular volume of tissue may be characterized as having a likelihood of responding to therapy or stimulation in a particular way. New and alternative methods and systems allowing probabilistic information to be relied upon when configuring and/or testing DBS are desired.

### Overview

The present inventors have recognized, among other things, that a problem to be solved is the need for new and/or alternative methods and systems allowing probabilistic information to be relied upon when configuring and/or testing DBS. In some examples, partitioning of brain structures around an implanted lead is performed using probabilistic shells.

A first illustrative and non-limiting example takes the form of a configuration system for configuring delivery of neuromodulation to specific tissue of a patient, the system comprising: a receiver module (400) configured to receive at least brain anatomy data for a patient and lead position data for a lead forming part of a neuromodulation system, the lead position data indicating a location of the lead in the brain of the patient; a structure selection module (404) coupled to a user interface (406) providing a graphical output allowing a user to identify and select brain structures in the patient's brain as target structures and as avoid structures; a voxel definition module (402) configured to define portions of the patient's brain in voxel form as a voxel data structure; wherein: the receiver module receives in the brain anatomy data a plurality of nested probability shells for a neural structure indicating a probability of a therapeutic outcome resulting from stimulation of volumes defined by the nested probability shells; and the voxel definition module is configured to determine voxel values for each voxel in the voxel data structure using the probability shells of the plurality of nested probability shells.

Additionally or alternatively, each probability shell has an outer border and defines an increase in probability relative to volumes outside the probability shell; and the voxel definition module is configured to determine voxel values for each voxel in the voxel data structure using the probability shells of the nested probability shells for the neural structure by: a) selecting a first probability shell; b) calculating, for the selected probability shell, a fill quantity for each voxel having at least a portion therein, the fill quantity representing a percentage of the voxel that is within the selected probability shell; c) multiplying, for each voxel in the selected probability shell, the fill quantity by the increase in probability of the selected probability shell, to yield a partial voxel value; repeating a), b), and c) for each probability shell of the nested probability shells for the neural structure.

Additionally or alternatively, the system further comprises an optimization block configured to determine optimal steering and amplitude settings for use by the neuromodulation system using data passed from the voxel definition module; wherein the voxel definition module is configured to pass a plurality of target shell and avoid shell structures to the optimization block, including one or more target shell or avoid shell structures generated by performing steps a), b) and c) for the selected probability shell of the nested probability shell of the neural structure.

Additionally or alternatively, each target shell or avoid shell structure comprises a plurality of partial voxel scores.

Additionally or alternatively, the voxel definition block is configured to sum the partial voxel values of each voxel to yield summed voxel values for each voxel relative to the neural structure, the system further comprising an optimization block configured to determine optimal steering and amplitude settings for use by the neuromodulation system using data passed from the voxel definition module; and the voxel definition module is configured to pass the summed voxel values for the neural structure.

Additionally or alternatively, each probability shell has an outer border and defines a probability applicable to volume within the probability shell that lies outside any further nested probability shell therein; and the voxel definition module is configured to determine voxel values for each voxel in the voxel data structure using the probability shells of the plurality of nested probability shells by, for each respective probability shell within which a voxel is at least partly located: a) calculating a partial fill representing a percentage of the voxel that is in the respective probability shell; b) calculating a partial voxel value by multiplying the partial fill by the probability for the respective probability shell; after completing a) and b) for each respective probability shell, summing all partial voxel values for each voxel, such that each voxel has a single summed voxel value relative to the nested probability shell for the neural structure.

Additionally or alternatively, the system further includes an optimization block configured to determine optimal steering and amplitude settings for use by the neuromodulation system using data passed from the voxel definition module; wherein the voxel definition module is configured to pass the summed voxel values to the optimization block.

Additionally or alternatively, each probability shell has an outer border and defines an increase in probability relative to tissue outside the probability shell; and the voxel definition module is configured to determine voxel values for each voxel in the voxel data structure using the probability shells of the plurality of nested probability shells by: selecting a first probability shell; calculating, for the selected probability shell, a fill quantity for each voxel having at least a portion therein, the fill quantity representing a percentage of the voxel that is within the selected probability shell; and calculating, for the selected probability shell, a shell weight by multiplying the increasing probability for the selected probability shell by a target or avoid structure weight received from a user via the structure selection block.

Additionally or alternatively, the system includes an optimization block configured to determine optimal steering and amplitude settings for use by the neuromodulation system using data passed from the voxel definition module; wherein the voxel definition module is configured to pass, for each nested probability shell, a set of voxel fill values and a shell weight.

Additionally or alternatively, the target structures correspond to beneficial therapeutic outcomes, and the avoid structures correspond to adverse therapeutic outcomes.

Additionally or alternatively, the optimization block includes a metric calculator is configured to determine each of: a target value calculated by determining a volume of activation of target structures for a selected steering configuration and amplitude; an avoid structure penalty calculated by determining a volume of activation of avoid structures for the selected steering configuration and amplitude; a background penalty calculated using a total volume of activation for the selected steering configuration and amplitude; and a metric as the target value less the avoid structure penalty and the background penalty.

Additionally or alternatively, the optimization block calculates the avoid structure penalty with a user-defined avoid structure weight, and the background penalty with a user-defined background ratio weight.

Additionally or alternatively, the system includes a therapy selection module and a communications module, the therapy selection module adapted to: present to a user at least one proposed therapy configuration for selection by the user; and in response to the user selecting a proposed therapy configuration for use, commanding the communications module to issue instructions to a pulse generator of the neuromodulation system to implement the selected proposed therapy configuration. Additionally or alternatively, the therapy selection module is configured to present to the user a graphic of a stimulation field model for the proposed therapy configuration.

A further example takes the form of a neuromodulation system comprising: a pulse generator; a lead configured for coupling to the pulse generator and adapted for positioning in a patient's brain; and a configuration system as in the preceding, wherein the pulse generator is adapted to receive the instructions from the therapy selection module and apply the selected proposed therapy configuration to the patient via the lead.

Another illustrative and non-limiting example takes the form of a method of configuring a neuromodulation system to deliver targeted to specific tissue of a patient, the method comprising: receiving at least brain anatomy data for a patient and lead position data for a lead forming part of the neuromodulation system, the lead position data indicating a location of the lead in the brain of the patient; identifying and selecting brain structures in the patient's brain as target structures and as avoid structures; defining portions of the patient's brain in voxel form as a voxel data structure; calculating, using the voxel data structure, at least one candidate therapy including optimized therapy parameters for use by the neuromodulation system including at least an amplitude for use in stimulation and electrode utilization data for use in stimulation; and selecting and communicating at least one candidate therapy to the neuromodulation system for using on the patient; wherein: the brain anatomy data includes a plurality of nested probability shells for a neural structure indicating a probability of a therapeutic outcome resulting from stimulation of volumes defined by the nested probability shells; and the step of defining portions of the patient's brain in voxel form includes determining voxel values for each voxel in the voxel data structure using the probability shells of the plurality of nested probability shells.

Additionally or alternatively, each probability shell has an outer border and defines an increase in probability relative to volumes outside the probability shell; and the step of defining portions of the patient's brain in voxel form includes determining voxel values for each voxel in the voxel data structure using the probability shells of the nested probability shells for the neural structure by: a) selecting a first probability shell; b) calculating, for the selected probability shell, a fill quantity for each voxel having at least a portion therein, the fill quantity representing a percentage of the voxel that is within the selected probability shell; c) multiplying, for each voxel in the selected probability shell, the fill quantity by the increase in probability of the selected probability shell, to yield a partial voxel value; repeating a), b), and c) for each probability shell of the nested probability shells for the neural structure.

Additionally or alternatively, the step of calculating, using the voxel data structure, at least one candidate therapy includes determining optimal steering and amplitude settings for use by the neuromodulation system using a plurality of target shell and avoid shell structures, including one or more target shell or avoid shell structures generated by performing steps a), b) and c) for the selected probability shell of the nested probability shell of the neural structure.

Additionally or alternatively, each target shell or avoid shell structure comprises a plurality of partial voxel scores.

Additionally or alternatively, the step of defining portions of the patient's brain in voxel form includes summing the partial voxel values of each voxel to yield summed voxel values for each voxel relative to the neural structure; and the step of calculating, using the voxel data structure, at least one candidate therapy includes using the summed voxel values.

Additionally or alternatively, each probability shell has an outer border and defines a probability applicable to volume within the probability shell that lies outside any further nested probability shell therein; and the step of defining portions of the patient's brain in voxel form includes determining voxel values for each voxel in the voxel data structure using the probability shells of the plurality of nested probability shells by, for each respective probability shell within which a voxel is at least partly located: a) calculating a partial fill representing a percentage of the voxel that is in the respective probability shell; b) calculating a partial voxel value by multiplying the partial fill by the probability for the respective probability shell; after completing a) and b) for each respective probability shell, summing all partial voxel values for each voxel, such that each voxel has a single summed voxel value relative to the nested probability shell for the neural structure.

Additionally or alternatively, the step of calculating, using the voxel data structure, at least one candidate therapy includes determining optimal steering and amplitude settings for use by the neuromodulation system using the summed voxel values.

Additionally or alternatively, each probability shell has an outer border and defines an increase in probability relative to tissue outside the probability shell; and the step of defining portions of the patient's brain in voxel form includes determining voxel values for each voxel in the voxel data structure using the probability shells of the plurality of nested probability shells by: selecting a first probability shell; calculating, for the selected probability shell, a fill quantity for each voxel having at least a portion therein, the fill quantity representing a percentage of the voxel that is within the selected probability shell; and calculating, for the selected probability shell, a shell weight by multiplying the increasing probability for the selected probability shell by a target or avoid structure weight received from a user via the structure selection block.

Additionally or alternatively, calculating, using the voxel data structure, at least one candidate therapy includes determining optimal steering and amplitude settings for use by the neuromodulation system using voxel fill values and a shell weight for each of the nested probability shells.

Additionally or alternatively, the target structures correspond to beneficial therapeutic outcomes, and the avoid structures correspond to adverse therapeutic outcomes.

Additionally or alternatively, calculating, using the voxel data structure, at least one candidate therapy includes: calculating a target value by determining a volume of activation of target structures for a selected steering configuration and amplitude; calculating an avoid structure penalty by determining a volume of activation of avoid structures for the selected steering configuration and amplitude; calculating a background penalty using a total volume of activation for the selected steering configuration and amplitude; and calculating a metric as the target value less the avoid structure penalty and the background penalty.

Additionally or alternatively, calculating the avoid structure penalty includes applying a user-defined avoid structure weight, and calculating the background penalty includes applying a user-defined background ratio weight.

Additionally or alternatively, the method also includes delivering, via the neuromodulation system, the selected candidate therapy to the patient.

Additionally or alternatively, the method also includes presenting, on a graphical user interface, a simulation of a stimulation field model for the candidate therapy.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative DBS system implanted in a patient;

FIG. 2 illustrates details of a directional DBS lead;

FIG. 3 shows an illustrative method in block form;

FIG. 4 shows an illustrative set of related probability shells;

FIG. 5 shows the probability shells of FIG. 4 against a partition grid;

FIG. 6 shows illustrative methods in block form; and

FIG. 7 shows an illustrative system.

### DETAILED DESCRIPTION

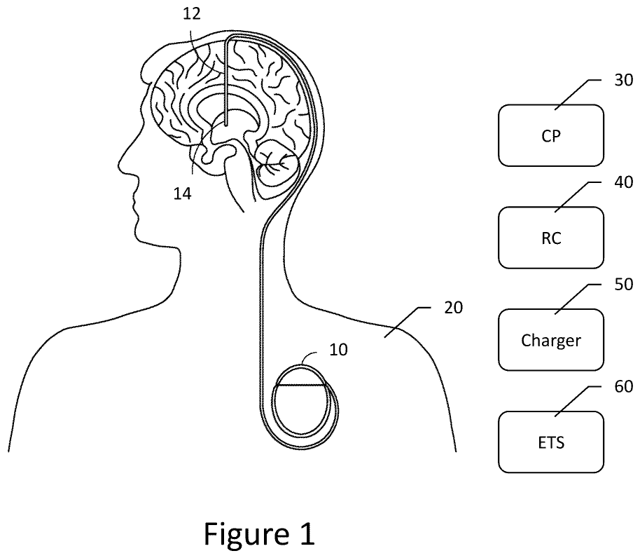

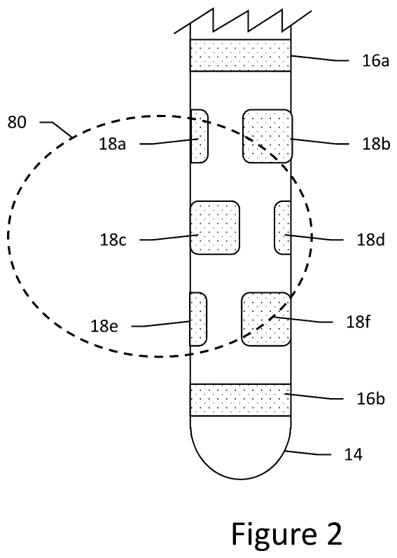

FIG. 1 shows an illustrative DBS system implanted in a patient. The system comprises a pulse generator 10, shown implanted in the pectoral region of a patient 20. The pulse generator 10 is coupled to a lead 12 which extends subcutaneously to the head of the patient 20, through a burr hole formed in the patient's skull, and then into the brain of the patient. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases.

DBS may be targeted, for example, and without limitation, at neuronal tissue in the thalamus, the globus pallidus, the subthalamic nucleus, the pedunculopontine nucleus, substantia nigra pars reticulata, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used herein are brain structures associated with therapeutic benefits, and avoidance regions or “Avoid” regions are brain structures associated with side effects.

Conditions to be treated may include dementia, Alzheimer's disease, Parkinson's disease, various tremors, depression, anxiety or other mood disorders, sleep related conditions, etc. Therapeutic benefits may include, for example, and without limitation, improved cognition, alertness, and/or memory, enhanced mood or sleep, avoidance of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 30 may be, for example, a laptop or tablet computer and can be used by a physician, or at the direction of a physician, to obtain data from and provide instructions to the pulse generator 10 via suitable communications protocols such as Bluetooth or MedRadio or other wireless communications protocols, and/or via other modalities such as inductive telemetry.

A patient remote control (RC) 40 can be used by the patient to perform various actions relative to the pulse generator 10. These may be physician defined options, and may include, for example, turning therapy on and/or off, entering requested information (such as answer questions about activities and therapy benefits and side effects), and making (limited) adjustments to therapy such as selecting from available therapy programs and adjusting, for example, amplitude settings. The RC 40 can communicate via similar telemetry as the CP 30 to control and/or obtain data from the pulse generator 10. The patient RC 40 may also be programmable on its own, or may communicate or be linked with the CP 30.

A charger 50 may be provided to the patient to allow the patient to recharge the pulse generator 10, if the pulse generator 10 is rechargeable. Some pulse generators 10 are not rechargeable, and so the charger 50 may be omitted. The charger 50 can operate, for example, by generating a varying magnetic field to activate an inductor associated with the pulse generator 10 to provide power to recharge the pulse generator, using known methods.

Some systems may include an external test stimulator (ETS) 60. The ETS 60 can be used intraoperatively to test therapy programs after the lead 12 has been positioned in the patient to determine whether therapy is or can work for the patient 20. For example, an initial implantation of the lead 12 can take place using, for example, a stereotactic guidance system, with the pulse generator 10 temporarily left out. The lead 12 may have a proximal end thereof connected to an intermediate connector (sometimes called an operating room cable) that couples to the ETS 60. After lead 12 has been implanted and coupled to the ETS 60, the ETS can be programmed using the CP 30 with various therapy programs and stimulation parameters, which are tested to determine therapy effects. Lead position may be adjusted, as needed during this process. Once therapy suitability for the patient is established, the permanent pulse generator 10 is implanted and the lead 12 is connected thereto, with the ETS then being removed from use.

The pulse generator 10 may include operational circuitry for generating output stimulation programs and/or pulses in accordance with stored instructions. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of the pulse generator 10, CP 30, RC 40, Charger 50, and ETS 60 can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

FIG. 2 illustrates details of a directional DBS lead. The distal end 14 is shown, and a plurality of electrodes are shown as well. Two ring electrodes 16a, 16b (collectively ring electrodes 16) can be provided as shown, and a number of segmented electrodes are shown at 18a, 18b, 18c, 18d, 18e, 18f (collectively, segmented electrodes 18). Each electrode 16, 18 is separately addressable in the system, such as by using a pulse generator having multiple independent current control (MICC), or multiple voltage sources,

MICC is a stimulus control system that provides a plurality of independently generated output currents that may each have an independent quantity of current. The use of MICC can allow spatially selective fields to be generated during therapy outputs. The term “fractionalization” may refer to how the total current issued by the pulse generator via the electrodes is divided up amongst the electrodes 16, 18 on the lead. It should be noted that the pulse generator canister may serve as an indifferent electrode or as a return electrode for therapy outputs; if desired, one of the electrodes (such as a ring electrode 16 or one or more of the segmented electrodes 18) may instead be used as a return electrode. Thus, for example, the electrodes on the lead may serve as cathodes while pulse generator canister serves as an anode during one phase of stimulation pulse delivery. In another example, some of the lead electrodes 16, 18 serve as cathodes, while other lead electrodes serve as anodes during one phase of stimulation pulse delivery. Any suitable combination and quantity of anodes and cathodes may be used for therapy purposes, and any lead electrode and/or the housing electrode can be used in any of these roles, as needed.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

MICC used with a directional lead can facilitate precise therapy targeting. For example, a directional lead as shown in FIG. 2 may be used to generate a stimulation field as illustrated at 80 in FIG. 2. The outer boundary of field 80 may be understood as representing an equipotential or equal field boundary, within which the electrical field is higher than an activation threshold, and outside of which the electrical field is below the threshold, for purposes of illustration. An activation threshold may represent or approximate a voltage/field threshold at which neural cells will activate or “fire”. Activation thresholds may be determined on a population basis, such as by relating to a voltage/field at which a 50% likelihood of activation of 50% of the cell population is determined, thought other boundaries/thresholds can be used. The shape of the field can be adjusted, as described variously in the references incorporated by reference above, by modifying the fractionalization of current issued via the electrodes using MICC. An output creating an activation field boundary as shown at 80 may be (roughly) generated by using electrode 18c as a cathode, and surrounding electrodes 18a, 18c, and 18d as anodes, for example. The actual characteristics of fractionalization may be more sophisticated than this simple example.

The boundary shown at 80 in FIG. 2 can be used to illustrate stimulation field effects, and can be generated for purposes of display using stimulation field modeling (SFM). In SFM, the tissue is modeled, for example, using finite element models in which the lead body is treated as an insulator, surrounded by a thin encapsulation sheath, and surrounded by neural tissue. The neural tissue may be modeled as isotropic and homogenous, though more sophisticated modelling can also be used if desired. A set of model voxels are defined around the lead, breaking up the volume into small segments, each of which can be analyzed within the model. The outer boundaries of the SFM can be determined using a population-based activation threshold as described above. The result can be that at a given fractionalization and total stimulation current, an SFM can be generated as a three-dimensional surface surrounding a portion of the lead and encompassing a volume of neural tissue. Field 80 may be, for example, understood as a two-dimensional representation of a slice of the SFM. As noted, the SFM can be used as a visual tool for illustrating, to a patient or physician, what tissue is or is not being stimulated by a given fractionalization and total current.

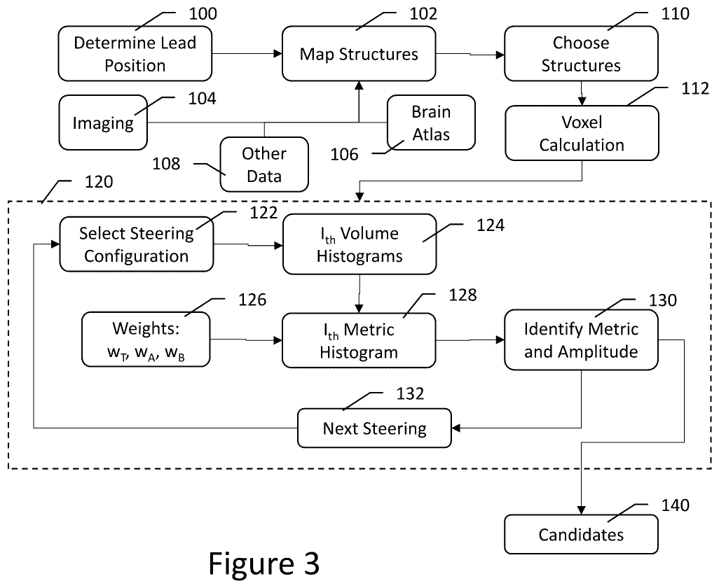

FIG. 3 shows an illustrative method in block form. In the illustrative method, lead position in the patient is determined at 100. For purposes of this illustration, lead position in the brain will be explained, however, the present invention may be used when modeling and optimizing neural stimulation in other parts of the body. The lead position 100 may be determined by use of imaging modalities such as X-ray, CT scan, MRI, or others, after the lead has been implanted.

Next, the system maps structures, as indicated at 102. Mapping structures 102 may include the use of pre-operative and/or post-operative imaging 104 and data from a brain atlas 106 to identify structures within the brain. A brain atlas 106 may include data from a population of patients indicating the general position and nature of structures in the brain, allowing the images to be referenced against population examples. Illustrative structures may include the thalamus, the globus pallidus, the subthalamic nucleus, and/or other structures listed above. Data input to 102 may also include other data 108 such as, for example and without limitation, inputs from a database of therapy settings of a previously programmed/treated population of patients, as desired. That is, therapy issued by other implantable systems may target similar and/or select structures repeatedly and so may provide additional understanding of, for example, best practices. Other data 108 may also include brain functional data such as gathered using functional-based imaging, or electrophysiological activities recorded using an implanted lead, each of which may also enter the system as data input at 102.

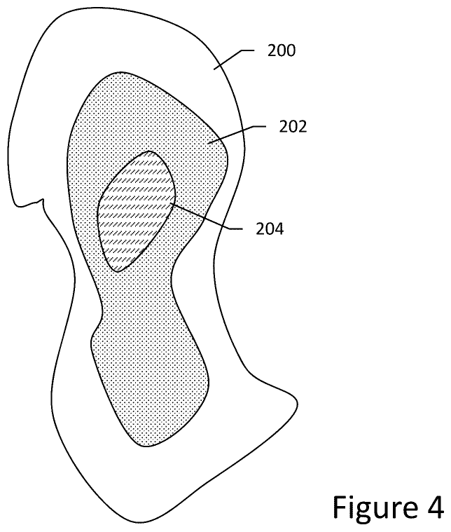

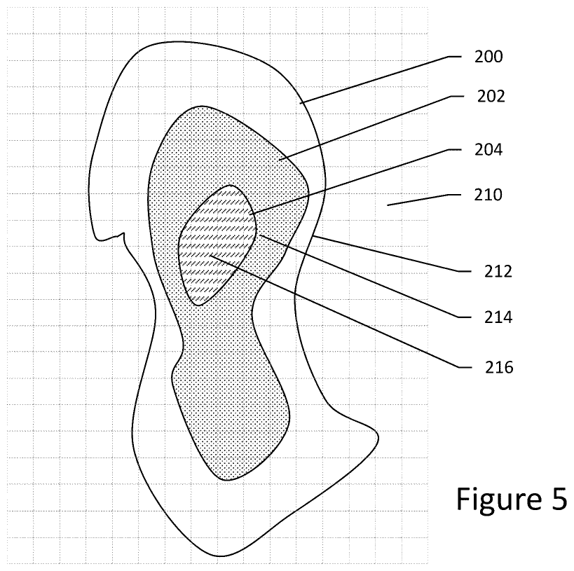

This collective data is used to map locations of structures in the brain, as well as “sweet” and “sour” spots-areas associated with therapeutic benefits and/or harms or side-effects. Prior systems have treated the identified brain structures at 102 as definitive, that is, treating particular locations and boundaries as the actual location of neural structures. Probability shells are further explained below, and the present invention modifies this prior approach by receiving and analyzing probability shells instead or in addition to other inputs. To this end, cross referencing the patient-specific imaging with a brain atlas and other data sources can allow a plurality of probability shells to be generated, as described further below relative to FIGS. 4 and 5.

The user or physician then chooses structures at block 110. Structures may be identified as target structures or avoid structures. Target structures are those that the physician determines should be stimulated as much as possible, and avoid structures are those that the physician determines should not be stimulated, to the extent possible. Typically, target structures are associated with therapy benefits, and avoid structures are those that are associated with side effects.

Voxel calculation occurs as indicated at 112. The voxel calculation 112 defines a grid of volume elements (voxels) in the tissue region around the lead, and determines which voxels are in various structures, as further described below. As used herein, “voxel” refers to any segment of volume used in an analysis, regardless of shape and may include cubes, polygons, partial cylinders, partial toroidal shapes, etc. For simplicity, the figures show volumes defined as cubes (as voxels may be defined in Cartesian coordinates), displayed in two dimensions. Voxel calculation may reference any of world, anatomical or image coordinate systems. Other voxel definitions and coordinate systems (such as spherical or polar coordinates) can be used if desired. Some systems may use an anatomical reference for the relevant coordinate system to define both lead position and structure positions/locations. The skilled person will understand transformations from one coordinate system to another. The voxel calculation 112 includes identification of which target and avoid structures contains which voxels, using the choices made at block 110. A single voxel may be in multiple structures.

An optimization follows in an iterative block 120. The structure choices 110 and voxel calculation 112, and/or device history or other inputs, are used to determine an initial steering configuration 122. The steering configuration 122 is then used to determine an Iₜₕ table. The contents of the Iₜₕ table indicate, for each voxel using a given steering state and fractionalization, the minimum total current that would be needed or likely to trigger neural activity in the given voxel. The values of the Iₜₕ table are used to create Iₜₕ Volume Histograms 124. For each target or avoid structure, an Iₜₕ Volume Histogram is created, and contains bins for each of the range of available amplitude settings (voltage or current levels). Each voxel is characterized as activated or not activated at a plurality of amplitudes, using the Iₜₕ table data. Relative to each target or avoid structure, the voxel has a “value” that is calculated as further discussed below, where the voxel value indicates, in part, the fraction of the voxel that is inside a particular structure. Each bin has a value and associated amplitude, where the bin value is determined by summing the product of the voxel volume, times voxel value, for each voxel that would be activated at the amplitude for the bin. That is, each bin in an Iₜₕ Volume Histogram specifies the change in stimulated volume of each structure at each of a range of amplitudes for the steering and other therapy parameters used to generate the Iₜₕ table.

The contents of these Iₜₕ Volume Histograms 124, and the target/avoid region selections, are combined with weights 126, to generate the Iₜₕ Metric Histogram 128. In a simple approach, there may be two user-adjustable weights, and one pre-set weight: target volume weight, w_{T} may be pre-set to 1, avoid structure weight, w_{A}, and background weight WB may be user adjustable; other approaches to weighting for target, avoid and background structures may be used. The Iₜₕ Metric Histogram 128 thus indicates, for each of a plurality of amplitudes, the resultant per-amplitude change in metric based on which voxels that are activated and within a target or avoid region, as weighted in accordance with weights 126.

The product of the weighting values 126 of each voxel and the Iₜₕ Volume Histograms 124 is referred to as an Iₜₕ Metric Histogram 128. The per-amplitude change in metric values are integrated to determine the highest metric value and the current amplitude that generates the highest metric value for the steering configuration under analysis, at block 130. These values, and those generated by previous iterations of the optimization, are used to generate a next steering state, prompting the next iteration, as indicated at 132. If an exit condition is met, such as by showing that the metric is not increasing with new steering configurations, the iterations in 120 terminate and the resulting candidates, including the various maximum metrics and amplitudes, are analyzed at 140.

During the analysis in FIG. 3, each voxel may be understood as having its own value, depending on whether the voxel is in a target region, avoid region, or background (neither target nor avoid). That value can then be used to generate the metric by multiplying with weight. At a high level, the total metric may be understood as indicated at Equation 1:

$$metric = \sum ( {v}_{target} - ( {v}_{avoid} * {w}_{A} ) - ( {v}_{sfm} * {w}_{B} ) ) \qquad Eq . 1$$

Where v_{target} is the volume of stimulated target tissue, v_{avoid} is the volume of the stimulated avoid region, and v_{sfm} is the total stimulated region at the optimized amplitude. In Equation 1, w_{A}, and we are as previously described, and each may be user adjustable; if desired, a target weight, w_{T}, (set to 1 and omitted in Equation 1) may be included and would multiply with v_{target}. An analogous version of Equation 1 may also be used on a voxel by voxel basis to populate the Iₜₕ Metric Histogram 128.

Further examples and possible details for use in the preceding description of the algorithm in FIG. 3 may be found in U.S. Pat. No. 11,195,609, the disclosure of which is incorporated herein by reference for details of a voxelization, histogram, and optimization procedure. However, the preceding description is one way that optimization 120 can be implemented. In other examples, a different order of operations may be used. For example, in an alternative, with each given steering configuration, a seeking algorithm may be used to test different amplitude measures without generating a histogram at all, with the seeking algorithm used through several iterations until a current amplitude at a given steering configuration is determined that maximizes the metric as calculated above in Equation 1.

While a simplest approach to finding highest metrics may be to sweep all available steering and amplitude configurations (along with other parameters such as pulse width, shape, frequency, etc.), the computational burden of such a procedure may be excessive. It may be preferable to use a more selective approach. In some examples, for a given patient's anatomy, lead position, and medical condition, the optimization at 120 may use similarity analysis to identify similar patient characteristics in a database, and may use such analysis to generate starting points for the optimization 120. As noted, these are merely examples.

Those combinations of steering configurations and stimulation parameters that yield highest metrics may be characterized as candidate therapies, as indicated at 140. Candidate therapies 140 may further be rated or analyzed using secondary factors, such as power consumption. The candidate therapies may be presented to the physician. The physician can then select steering configurations and stimulation parameters for use in subsequent testing of the system. Testing can occur using an ETS or implantable pulse generator, as desired.

As noted, the preceding discussion of FIG. 3 is simplified and does not address the use of probability shells generated as part of the map structures process 102. Rather than a determinative output from the map structures process 102, a set of probability shells can be generated, providing richer detail and acknowledging that the actual position of the structures to be targeted and avoided cannot be known with exact precision from the combination of imaging and brain atlas data. Moreover, not just overall structures can be defined, but also regions within a given structure that is to be targeted, such as targeting an anterior portion or a posterior portion of a given structure.

FIG. 4 shows an illustrative set of related probability shells, shown as a cross section of 3-dimensional shells. In an example, each of the shells indicates a probability that a particular structure is present inside a given border or, alternatively, that a stimulus issued to a location inside a given border has a likelihood of causing a neural response, whether desired or undesired. For example, the outer probability shell 200 may indicate a 10% likelihood that stimulation delivered within the shell 200 (and outside the next shell 202) will yield a therapeutic result. The next probability shell 202 may indicate an analogous 20% likelihood, and shell 204 may indicate a 50% likelihood. The set of shells 200, 202, 204 may be described as a nested set of probability shells, all indicating a likelihood that stimulation delivered to the interior of the shell will yield a therapeutic result. If the shells 200, 202, 204 all related to a target region, then a beneficial therapeutic result is expected.

FIG. 5 shows the probability shells of FIG. 4 against a voxel grid, again shown as a cross section of 3-dimensional shells. The grid defines a plurality of individual voxels 210. During the process of voxelization, each voxel is analyzed by review of both the border and meaning of each probability shell, to yield a value for the voxel. Several approaches can be used.

In a first example, separate voxelizations are generated for each probability shell, and each of these voxelizations correspond to either target or avoid regions. Thus, first, second and third voxelizations are generated and stored, corresponding to shells 200, 202 and 204, with each voxelization storing a percentage value equal to the increase in probability represented by each subsequent voxel. In later discussion, this can be termed “Unweighted Multiple Targets.” Thus, the first voxelization has a probability of 10% (10% minus zero), the second voxelization has an increase in probability of 10% (20% minus 10%), and the third voxelization has an increase in probability of 30% (50% minus 20%). Assuming the nested probability shells 200, 202, 204 are for a target structure as identified by the physician:
Voxel 210 is outside the each of the probability shells 200, 202, 204, and has a target value of 0 for each of the first, second and third voxelizations.
Voxel 212 is 60% inside shell 200, and entirely outside shells 202 and 204, and has a target value of 0.6 for the first voxelization, and 0 for each of the second and third voxelizations.
Voxel 214 is 30% inside 204, is entirely inside shells 200 and 202, and has a value of 1 for each of the first and second voxelizations, and a value of 0.3 for the third voxelization.
Voxel 216 is entirely within each of shells 200, 202, and 204, and has a value of 1 for each voxelization.

In the illustration, any time a voxel is identified having multiple values, each such value may be considered a “partial voxel value.” In some examples, the partial voxel values are summed together prior to passing to an optimization module or step, and in others each partial voxel value is passed on instead of being summed. As noted, each of the three voxelizations in the above example carries a stepped probability value, representing the increase of probability that the shell represents. Thus “Unweighted Multiple Targets” are defined. Each shell can then be passed on as a separate target in the subsequent metric calculation. Because each shell represents a probability, the weight associated with each shell can be discounted. For example, if each shell is part of a structure given a weight of 2, the first voxelization would be treated as a first target structure with a weight of 2*0.10 (the weight times the probability), or 0.2; the second shell would also have a weight of 0.2, while the third shell would have a weight of 0.6. The downside then is that target-specific weights need to be recalculated in the voxelization, and then transmitted to the iterative analysis at 120 of FIG. 3.

A next example may be described as using “Weighted Multiple Targets.” In this example, the voxel data for each voxelization can carry the probability of a respective shell, thereby weighting the targets according to probability. Here, for example:
Voxel 210 is outside the each of the probability shells 200, 202, 204, and has a target value of 0 for each of the first, second and third voxelizations.
Voxel 212 is 60% inside shell 200, and entirely outside shells 202 and 204, and has a target value of 0.06 (60% times 10%) for the first voxelization, and 0 for each of the second and third voxelizations.
Voxel 214 is 30% inside 204, is entirely inside shells 200 and 202, and has a target value of 0.1 (1 times 10%) for each of the first and second voxelizations, and 0.09 (30% times 30%) for the third voxelization.
Voxel 216 is entirely within each of shells 200, 202, and 204, and has a target value of 0.1 for each of the first and second voxelizations, and 0.3 for the third voxelization.

Each voxelization would be passed on as first, second and third target structures, however, because the voxel values calculated above account for probability of each shell, there is no need to discount the weight of each voxelization as with the Unweighted Multiple Targets. Here then, the process uses Weighted Multiple Targets, and the use of the weighting from the user in the optimizer is unchanged. Instead, each voxelization and resultant target structure would have the same weight in the optimizer, using whatever weight is assigned by the user in the structure choice step 110 of FIG. 3.

In the previous two examples, the voxel calculation 112 can pass forward multiple targets, where each of the first, second and third voxelizations described above are passed on to block 120. For example, a given voxel may be processed multiple times during the optimization process as each target structure is separately analyzed. Doing so will increase the amount of analysis performed at each iteration of block 120, particularly at block 128.

In another approach, the values calculated for a particular voxel in the first, second, and third voxelizations can be summed together into a single voxel data structure and a single target structure would be passed forward to block 120. In so doing, again using the above numerical example, the resultant single voxel structure would have these values:
Voxel 210 would have a resultant target value of 0+0+0=0
Voxel 212 would have a resultant target value of 0.06+0+0=0.06
Voxel 214 would have a resultant target value of 0.1+0.1+0.09=0.29
Voxel 216 would have a resultant target value of 0.1+0.1+0.3=0.5
   Because the maximum theoretical probability would be 1.0 for a probability shell, the maximum target value for any voxel would also be 1.0 using this approach. Because both probability and fractional fill information is included in the resulting target structure voxel values, the structure weight assigned by the user at 110 in FIG. 3 can be applied without any modification needed when executing step 128. This process may be referred to as “Single Target Summation” for reference purposes herein, as the summation occurs at the end of the procedure.

Returning again to FIG. 4, another illustrative example performs the voxelizing steps by directly incorporating probabilities at the outset. Using the previous example of region 200 representing a 10% probability shell, with region 202 representing a 20% probability shell, and region 204 representing a 50% probability shell, the probabilities can be directly incorporated in voxelizing. The following analysis can be performed:
Voxel 210 is outside the each of the probability shells 200, 202, 204, and has a target value of 0.
Voxel 212 is 60% inside shell 200, and entirely outside shells 202 and 204, and has a target value of 0.06 (60% times 10%).
Voxel 214 is 30% inside 204, is entirely inside shells 200 and 202, and has a target value that is the sum of 70% times 20% (for the portion outside shell 204 and inside shells 200 and 202) plus 30% times 50% or 0.14+0.15=0.29.
Voxel 216 is entirely within each of shells 200, 202, and 204, and has a target value of 0.5, which is the product of the highest applicable probability shell times one.
   This may be referred to as an “Integrated Single Target,” as the integration is occurring at the outset of the voxel definition. The resulting target structure data is the same as with Single Target Summation, meaning structure weight need not be adjusted, but the order and number of steps is different. The Integrated Single Target procedure allows calculating one voxelization, rather than multiple, possibly requiring less memory than Single Target Summation, but using more complex operations.

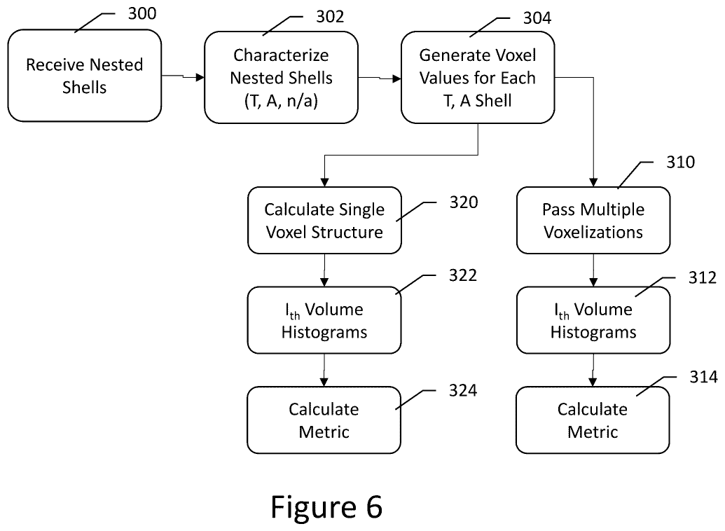

FIG. 6 shows illustrative methods in block form. Two alternative methods are shown in the figure, but each starts with the same basic steps, as nested shells are received at 300, and characterized 302 as target (T), avoid (A), or, optionally, as not relevant (n/a). Here, the physician may be shown, via a user interface, a plurality of structures that are near the implanted lead, and the physician identifies those structures as target or avoid structures. If the physician does not identify a particular structure as target or avoid, then it may go into the not relevant class, and is treated as background. Voxel values are generated for each of the target (T) and avoid (A) shells at block 304. In block 304, either a single voxel structure is generated (such as by Single Target Summation or Integrated Single Target analysis) and passed on at 320, or multiple voxelizations (such as by Weighted Multiple Targets or Unweighted Multiple Targets) are passed, as indicated at 310.

If multiple voxelizations are passed at 310, there are two ways of doing so. With the Weighted Multiple Targets, a plurality of targets are passed forward, each with its own weight, as described above. With Unweighted Multiple Targets, plural targets are passed to the optimizer, which uses the same weight for each during the optimizing procedure. The multiple voxelizations can be passed, as indicated at 310, to further analysis as separate target structures, which may or may not overlap. This means that the Iₜₕ Volume Histograms generated at 312 would include an Iₜₕ Volume Histogram for each voxelization. If the separate target structures overlap, one voxel may be processed multiple times during block 312, which can be the case in any event as a single voxel may be part of multiple structures. The analytical results for each target are summed as part of the metric calculation as indicated at 314, after each Iₜₕ Volume Histogram is calculated. With Weighted Multiple Targets, block 314 would reference a different weight for each of the separate targets associated with a given structure; the Unweighted Multiple Targets, block 314 could refer to the same weight for each target associated with the structure. With each of the multiple target approaches passing via 310/312/314, the number of calculations during the optimization, as well as the amount of memory needed for the additional histograms, may add to the processing power needed.

On the other hand, a single voxel structure may be calculated as indicated at 320. This may include the Single Target Summation or Integrated Single Target analysis described above. It may be that fewer and simpler floating-point operations would be needed for these configurations within the optimizer, as fewer voxel structures are used when generating the Iₜₕ Volume Histograms at block 322. The metric for this single target, as well as the rest of the Iₜₕ Volume Histograms, can be calculated as indicated at 324. The single target approaches may require extra analysis before passing the target to the optimizer (block 120 of FIG. 3), but are simpler to process once in the optimization.

In each of the above examples referencing shells 200, 202, and 204, the same analysis may apply in the event that the physician identifies an avoid structure rather than a target structure as corresponding to the shells 200, 202 and 204.

In an alternative, avoid structures may be treated differently than target structures by ignoring the probabilities entirely, and treating all shells that represent a probability above a predetermined threshold as having a probability of 1. For example, any shell with a probability equal to or greater than 10% may be treated as having a probability of 1; if so, assuming again that shell 200 represents 10% probability, and shells 202 and 204 represent greater probabilities, then all the shells 200, 202 and 204 can be treated as having probability of 1. If desired, the fill ratio of individual voxels may still be used so that the avoid voxels can have a value of 1 if entirely inside any of shells 200, 202 and 204, a value of 0 if outside all of the shells, and a value equal to the fill ratio. Alternatively, for any voxels having any portion inside an avoid shell, the fill ratio may be treated as 1. Various combinations of these analyses can be envisioned.

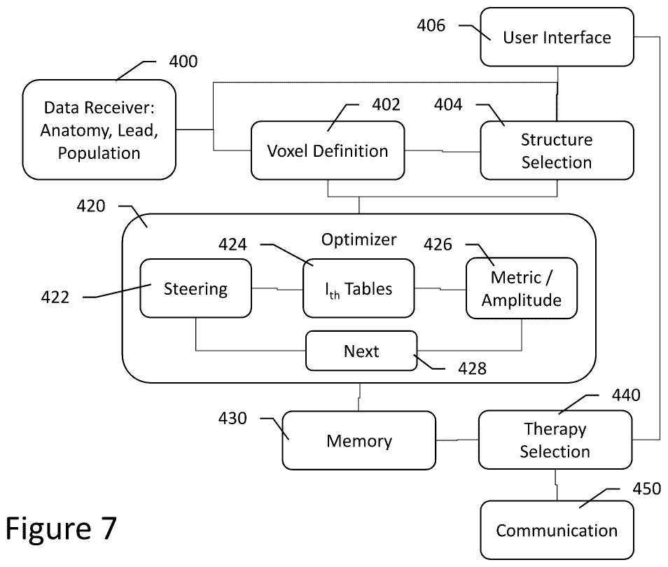

FIG. 7 shows an illustrative system. The system may include a data receiver block 400. For example, a communication circuit 450 may be coupled to a software or hardware module that is configured to receive and store information related to the patient anatomy, lead position, and/or other information including, for example, population-based structure data. Data input to 400 may also include inputs from a database of therapy settings of a previously programmed/treated population of patients, as desired. Also, brain functional data such as gathered using functional-based imaging, or electrophysiological activities recorded using an implanted lead, may also be used as data input at 400 as part of the overall mapping of “sweet” and “sour” spots-areas associated with therapeutic benefits and/or harms or side-effects. The data in block 400 is then used for voxel definition at 402, which may be implemented as another software module and/or by a separate or dedicated circuit (application specific integrated circuit, microcontroller, etc.). The voxel definition block 402 provides a voxelization to a structure selection block 404.

The structure selection block 404 is configured to receive user/physician inputs from a user interface 406, which may include one or more of a keyboard, mouse, trackball, touchscreen, monitor/output screen, voice or other audio input/output devices, etc. The structure selection block 404 may also receive structure data, such as would be helpful to identify structure boundaries as well as to identify structures, from the data receiver 400. The user interface 406 allows the user/physician to identify and select target and avoid regions in the patient anatomy for use by the structure selection block 404. If desired, block 404 may also limit which structures can be selected as targets, given a clinician's intended treatment and/or labeling limitations of the system.

The user interface is also used to allow the user/physician to select or modify the background, target, and/or and avoid structure weights that are used as illustrated above. Structure selection 404 may be applied to the voxel definitions to identify, on a structure-by-structure basis, the priority of stimulating (using for example, separate weights for each target structure) and/or avoiding (using for example, separate weights for each avoid structure) each structure. The effect of structure selection 404 is then to use the voxelization to determine the value of each voxel for each target and avoid structure. As used herein, the value of a voxel is as described above, to indicate each of the quantity of voxel fill by structures or portions of structures, such as probability shells, and probability of a neural response occurring if stimulated, and/or presence of a structure (whether target or avoid) in the voxel. The target and avoid structure data, along with voxel values corresponding thereto, are passed to the optimizer 420.

In some examples, the data going to block 420 can be any of the above discussed voxelizations, including, for example, single or multiple target/avoid structure data corresponding to nested probability shells, such as the Single Target Summation, Integrated Single Target, Weighted Multiple Targets, and Unweighted Multiple Targets. The optimizer 420 starts a series of iterative analyses with selecting a steering configuration at 422. The Iₜₕ tables are generated at 424, and metric/amplitude data is generated at 426. That is, the metric/amplitude data provides an indication, for a given steering configuration, of the pairings of metrics and amplitudes that would result. One or more best combinations are selected and stored to memory 430 by the optimizer 420. Absent exit conditions occurring, a next iteration is triggered at 428, and a new steering configuration is set at 422 and the process continues to iterate. Exit conditions and steering reconfiguration selections may be as described, for example, in U.S. Pat. No. 11,195,609, the disclosure of which is incorporated herein by reference.

When exit conditions are met, the set of data in memory 430 will provide one or more “best” or highest scoring steering settings and amplitude or parameter selections. These are then presented by a therapy selection module 440 to the user/physician via the user interface 406. The user/physician may then select or approve one or more proposed therapy. Therapy selection block 440 may generate an SFM for display via the user interface 406 to aid in the therapy selection process. Once a user has selected a therapy for implementation, a communications block 450 is used to communicate the therapy to a pulse generator or ETS. The pulse generator or ETS then delivers the selected therapy to the patient.

Steering selections at 422 may use an artificial intelligence method and/or a seeking function, with stop conditions predetermined. Block 420 may be configured to receive structure selection data that can inform the process of choosing steering configurations, in an example. For example, steering choices for the iterative process may use, without limitation, a database of steering selection configurations used for other, similar patients. By generically comparing the database of steering configurations to received structure selections, the optimizer 420 and steering selection block 422 may be able to eliminate a large portion of possible steering configurations quickly, to construct a shortened list. Some illustrative methods include iterative optimization methods such as gradient descent search, genetic algorithms, simulated annealing, random coordinate descent, particle swarm, fuzzy logic, among other machine learning search algorithms. Various details of the searching process are explained as well in U.S. Pat. No. 11,195,609, the disclosure of which is incorporated herein by reference.

While much of the above discussion focuses on use for DBS, other tissue regions can also be treated. For example, anatomical mapping may be used to identify neural and/or other structures to be targeted or avoided during other therapy, such as spinal cord stimulation (SCS), peripheral nerve stimulation, occipital nerve stimulation, muscles and muscle nerve fibers, therapies directed to the digestive tract, or other regions, and vagus nerve stimulation, As an example, in SCS, for example in the cervical spine, the spinal cord carries neural signals from various different parts of the body. As the science relating to spinal cord structure advances, knowledge may be obtained as to which parts of the spinal cord at a given vertebral level carry signals to and from what part of the body. With this knowledge, therapy and avoid regions in the spinal cord may be defined/determined. If a plurality of leads are present, or a paddle lead, at the given vertebral level, a steering and SFM model may be determined for the given vertebral level using a spinal cord atlas as well as lead implantation/position data (such as from an X-ray or other imaging system). A similar process as just described may be used to define therapy that targets the portion of the spinal cord carrying neural signals (such as pain signaling) that are to be interfered with, while other portions (such as carrying motor signals) are to be avoided. The metric calculations described above can then be used to optimizer steering to limit side effects and achieve desired therapy.

Turning back to FIG. 7, data receiver 400 may include a communications circuit (a transceiver, antenna or the like, for example, Bluetooth, WiFi, Medradio, etc.) and/or inputs/output circuits for use with, for example, local area network cables. In some examples, on the other hand, the data receiver 400 is instead a software module that communicates with other applications in the CP; communication via external hardware can be optional for data receiver 400. A microcontroller or microprocessor with particularly configured software or other instructions stored therewith, for example on non-transient memory, such as memory 430 which may include Flash, RAM, ROM, etc., as are known in the art. Overall the implementation in FIG. 7 may be on a table or laptop computer, such as a clinician programmer or CP as described above.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A configuration system for configuring delivery of neuromodulation to specific tissue of a patient, the system comprising:
a receiver module configured to receive at least brain anatomy data for a patient and lead position data for a lead forming part of a neuromodulation system, the lead position data indicating a location of the lead in the brain of the patient;
a structure selection module coupled to a user interface providing a graphical output allowing a user to identify and select brain structures in the patient's brain as target structures and as avoid structures;
a voxel definition module configured to define portions of the patient's brain in voxel form as a voxel data structure; wherein:
the receiver module receives in the brain anatomy data a plurality of nested probability shells for a neural structure indicating a probability of a therapeutic outcome resulting from stimulation of volumes defined by the nested probability shells; and
the voxel definition module is configured to determine voxel values for each voxel in the voxel data structure using the probability shells of the plurality of nested probability shells.

2. The configuration system of claim 1, wherein:
each probability shell has an outer border and defines an increase in probability relative to volumes outside the probability shell; and
the voxel definition module is configured to determine voxel values for each voxel in the voxel data structure using the probability shells of the nested probability shells for the neural structure by:
a) selecting a first probability shell;
b) calculating, for the selected probability shell, a fill quantity for each voxel having at least a portion therein, the fill quantity representing a percentage of the voxel that is within the selected probability shell;
c) multiplying, for each voxel in the selected probability shell, the fill quantity by the increase in probability of the selected probability shell, to yield a partial voxel value;
repeating a), b), and c) for each probability shell of the nested probability shells for the neural structure.

3. The configuration system of claim 2, further comprising an optimization block configured to determine optimal steering and amplitude settings for use by the neuromodulation system using data passed from the voxel definition module; wherein the voxel definition module is configured to pass a plurality of target shell and avoid shell structures to the optimization block, including one or more target shell or avoid shell structures generated by performing steps a), b) and c) for the selected probability shell of the nested probability shell of the neural structure.

4. The configuration system of claim 3, wherein the optimization block includes a metric calculator configured to determine each of:
a target value calculated by determining a volume of activation of target structures for a selected steering configuration and amplitude;
an avoid structure penalty calculated by determining a volume of activation of avoid structures for the selected steering configuration and amplitude
a background penalty calculated using a total volume of activation for the selected steering configuration and amplitude; and
a metric as the target value less the avoid structure penalty and the background penalty.

5. The configuration system of claim 4, wherein the optimization block calculates the avoid structure penalty with a user-defined avoid structure weight, and the background penalty with a user-defined background ratio weight.

6. The configuration system of claim 3, wherein each target shell or avoid shell structure comprises a plurality of partial voxel scores.

7. The configuration system of claim 2, wherein the voxel definition block is configured to sum the partial voxel values of each voxel to yield summed voxel values for each voxel relative to the neural structure, the system further comprising an optimization block configured to determine optimal steering and amplitude settings for use by the neuromodulation system using data passed from the voxel definition module; and the voxel definition module is configured to pass the summed voxel values for the neural structure.

8. The configuration system of claim 1, wherein:
each probability shell has an outer border and defines a probability applicable to volume within the probability shell that lies outside any further nested probability shell therein; and
the voxel definition module is configured to determine voxel values for each voxel in the voxel data structure using the probability shells of the plurality of nested probability shells by, for each respective probability shell within which a voxel is at least partly located:
a) calculating a partial fill representing a percentage of the voxel that is in the respective probability shell;
b) calculating a partial voxel value by multiplying the partial fill by the probability for the respective probability shell;
after completing a) and b) for each respective probability shell, summing all partial voxel values for each voxel, such that each voxel has a single summed voxel value relative to the nested probability shell for the neural structure.

9. The configuration system of claim 8, further comprising an optimization block configured to determine optimal steering and amplitude settings for use by the neuromodulation system using data passed from the voxel definition module; wherein the voxel definition module is configured to pass the summed voxel values to the optimization block.

10. The configuration system of claim 1, wherein:
each probability shell has an outer border and defines an increase in probability relative to tissue outside the probability shell; and
the voxel definition module is configured to determine voxel values for each voxel in the voxel data structure using the probability shells of the plurality of nested probability shells by:
selecting a first probability shell;
calculating, for the selected probability shell, a fill quantity for each voxel having at least a portion therein, the fill quantity representing a percentage of the voxel that is within the selected probability shell; and
calculating, for the selected probability shell, a shell weight by multiplying the increasing probability for the selected probability shell by a target or avoid structure weight received from a user via the structure selection block.

11. The configuration system of claim 10, further comprising an optimization block configured to determine optimal steering and amplitude settings for use by the neuromodulation system using data passed from the voxel definition module; wherein the voxel definition module is configured to pass, for each nested probability shell, a set of voxel fill values and a shell weight.

12. The configuration system of claim 1, wherein the target structures correspond to beneficial therapeutic outcomes, and the avoid structures correspond to adverse therapeutic outcomes.

13. The configuration system of claim 1, further comprising a therapy selection module and a communications module, the therapy selection module adapted to:
present to a user at least one proposed therapy configuration for selection by the user; and
in response to the user selecting a proposed therapy configuration for use, commanding the communications module to issue instructions to a pulse generator of the neuromodulation system to implement the selected proposed therapy configuration.

14. The configuration system of claim 13, wherein the therapy selection module is configured to present to the user a graphic of a stimulation field model for the proposed therapy configuration.

15. A neuromodulation system comprising:
a pulse generator;
a lead configured for coupling to the pulse generator and adapted for positioning in a patient's brain; and
a configuration system as in claim 13, wherein the pulse generator is adapted to receive the instructions from the therapy selection module and apply the selected proposed therapy configuration to the patient via the lead.

16. A method of configuring a neuromodulation system to deliver targeted to specific tissue of a patient, the method comprising:
receiving at least brain anatomy data for a patient and lead position data for a lead forming part of the neuromodulation system, the lead position data indicating a location of the lead in the brain of the patient;
identifying and selecting brain structures in the patient's brain as target structures and as avoid structures;
defining portions of the patient's brain in voxel form as a voxel data structure;
calculating, using the voxel data structure, at least one candidate therapy including optimized therapy parameters for use by the neuromodulation system including at least an amplitude for use in stimulation and electrode utilization data for use in stimulation; and
selecting and communicating at least one candidate therapy to the neuromodulation system for using on the patient; wherein:
the brain anatomy data includes a plurality of nested probability shells for a neural structure indicating a probability of a therapeutic outcome resulting from stimulation of volumes defined by the nested probability shells; and
the step of defining portions of the patient's brain in voxel form includes determining voxel values for each voxel in the voxel data structure using the probability shells of the plurality of nested probability shells.

17. The method of claim 16, wherein:
each probability shell has an outer border and defines an increase in probability relative to volumes outside the probability shell; and
the step of defining portions of the patient's brain in voxel form includes determining voxel values for each voxel in the voxel data structure using the probability shells of the nested probability shells for the neural structure by:
a) selecting a first probability shell;
b) calculating, for the selected probability shell, a fill quantity for each voxel having at least a portion therein, the fill quantity representing a percentage of the voxel that is within the selected probability shell;
c) multiplying, for each voxel in the selected probability shell, the fill quantity by the increase in probability of the selected probability shell, to yield a partial voxel value;
repeating a), b), and c) for each probability shell of the nested probability shells for the neural structure.

18. The method of claim 17, wherein the step of calculating, using the voxel data structure, at least one candidate therapy includes determining optimal steering and amplitude settings for use by the neuromodulation system using a plurality of target shell and avoid shell structures, including one or more target shell or avoid shell structures generated by performing steps a), b) and c) for the selected probability shell of the nested probability shell of the neural structure.

19. The method of claim 18, wherein each target shell or avoid shell structure comprises a plurality of partial voxel scores.

20. The method of claim 17, wherein the step of defining portions of the patient's brain in voxel form includes summing the partial voxel values of each voxel to yield summed voxel values for each voxel relative to the neural structure; and the step of calculating, using the voxel data structure, at least one candidate therapy includes using the summed voxel values.
